(19) United States
(12) Patent Application Publication
(10) Pub. No.: US 2025/0235188 A1
(43) Pub. Date: Jul. 24, 2025

(54) SALIVA COLLECTION DEVICES AND METHODS

(71) Applicant: LIFE TECHNOLOGIES CORPORATION, Carlsbad, CA (US)

(72) Inventors: Laura CHAPMAN, Carlsbad, CA (US); Alexandre VLASSOV, Carlsbad, CA (US); Tommie JACKSON, Carlsbad, CA (US); Aaron RICICA, Carlsbad, CA (US); Roy THOMPSON, Carlsbad, CA (US)

(21) Appl. No.: 18/853,509

(22) PCT Filed: Apr. 2, 2023
(86) PCT No.: PCT/US2023/017232

§ 371 Date: Oct. 2, 2024

Related U.S. Application Data
(60) Provisional application No. 63/327,104, filed on Apr. 4, 2022.

(51) Int. Cl. A61B 10/00; G01N 1/34

(52) U.S. Cl. CPC A61B 10/0051; G01N 1/34

(57) ABSTRACT

Systems and methods under the present disclosure include sample collection kits, such as for saliva or other fluids. In certain embodiments a detachable funnel can collect saliva from a user. A debris filter can be coupled to the funnel that extends into the test tube. The funnel can comprise or be coupled to a reagent cartridge that, when manipulated by a user, can break open and release reagent into the test tube for use in testing the saliva. Certain embodiments can comprise a casing that can house a test tube and tube cap, with a funnel configured to cover the entire top edge of the casing so as to protect the test tube and tube cap until used by a user.

### TECHNICAL FIELD

This disclosure generally relates to saliva collection devices and kits.

### RELATED TECHNOLOGY

Saliva is rapidly becoming a very common sample type for diagnostic and genealogy studies. Over the last two years more samples are collected at home rather than at a medically supervised facility. This means that the saliva collection device must be extremely easy to use and understood by a wide range of ages, intellectual abilities and physical abilities. This device—which in some cases is an empty device or it might contain transport media—should be also safe for at home use and transportation. The device should be easy to pack and ship to an analytical lab for analysis. And finally, the saliva collection device must be compatible with standard lab equipment for the most efficient analysis workflows-which involve analysis of DNA, RNA, protein or small molecules derived from saliva.

Saliva collection is much easier and painless for individuals versus common nasopharyngeal (NP) swabs for respiratory virus detection, such as SARS-COV-2. For this reason, many people prefer to collect saliva than have an NP swab. NP swabs are especially difficult for the young and elderly, who may have more difficulty to sit still or in a position for an NP swab. The other benefit of saliva is that it can be collected at home and a medical professional is not required to collect the sample. Accordingly, a saliva collection device and process needs to be easy to use for all ages and abilities. Furthermore, once the saliva sample arrives at a lab for analysis, the sample collection device needs to be easy to open and process, and compatible with standard laboratory equipment.

There are several saliva collection devices currently on the market for purchase, however none of these are very user friendly for at home collection. Typically, there are multiple parts in these devices that a user has to assemble in a correct orientation for the saliva to collect properly. Furthermore, once the collection tubes arrive at a lab for analysis, some of these devices are incompatible with standard lab equipment, such as liquid handlers and tube racks. One current saliva collection device (Spectrum™) has a false bottom on the collection tube, making the tube top heavy and prone to falling over. This device also has a cap design that contains the inactivation/stabilization reagent. However, the cap design is not compatible with automated decappers. The cap also requires a lot of force from a user to screw the cap on to fully release the reagent from the cap. Further, because the cap is perforated, liquid can be both in the bottom of the tube and in the cap, which makes it very challenging to ensure homogenous mixing of the inactivation reagent with the saliva. Another saliva collection device (Isohelix™) has a funnel design to prevent spillage if the tube should fall over while collecting saliva. However, this design slows the saliva going into the tube, thus taking longer to collect the saliva sample. Yet another collection device (DNA Genotek™), has the inactivation/stabilization reagent in an attached funnel lid and a false bottom on the collection tube. This makes the design extremely top heavy and very prone to tipping over. Once a user has collected saliva, the user must close the funnel lid to break open the seal for the reagent to flow into the tube. This can lead to splashing of the liquid out of the funnel as the lid is snapped close. Once the reagent is in the tube with the saliva, a standard size cap is screwed onto the tube and is ready to go to the lab for analysis.

Existing devices are plagued by one or more problems, such as, require assembly by a user, and/or difficult to release a stabilization reagent, and/or top heavy and prone to tipping over and spilling saliva everywhere, and/or have complex instructions unsuitable for general public use, and/or incompatible with lab processing. Hence, there is still no saliva collection device on the market that is user friendly and compatible with downstream lab analysis.

### BRIEF SUMMARY

One embodiment of the present disclosure comprises a sample collection kit for a standard specimen tube and standard cap. A kit of the disclosure comprises a funnel detachably coupled to the specimen tube and configured to direct test material in the test tube; and a reagent cartridge configured to comprise a reagent wherein the reagent cartridge can be coupled to the funnel. In some embodiments, the funnel can comprise one or more protrusions on the funnel that are configured to puncture the reagent cartridge when the reagent cartridge is manipulated by a user such that the reagent will pass into the test tube.

Another embodiment is a sample collection kit for a specimen tube. The sample collection kit can comprise a funnel configured to receive a sample from a user; wherein the funnel is configured to be attachable to a specimen tube.

Another embodiment is another sample collection kit for a specimen tube. It can comprise a casing configured to contain a specimen tube, a top edge of the casing comprising an opening configured to receive the specimen tube therein and a coupler configured to hold a tube cap.

Another embodiment is a sample collection kit for a specimen tube. The kit can comprise a funnel detachably coupled to the specimen tube and configured to direct test material into the specimen tube; a reagent cartridge configured to contain a reagent and to be coupled to the funnel; and one or more protrusions on the funnel configured to puncture the reagent cartridge when the reagent cartridge is manipulated by a user such that the reagent will pass into the specimen tube; wherein the funnel and one or more protrusions comprise a funnel apparatus configured to attach to a top edge of a casing, and further configured to cover an opening for a specimen tube and a coupler for a tube cap on the top edge.

A further embodiment under the present disclosure is a method for collecting and testing a sample. Steps of the method include receiving a sample in a funnel and directing the sample from the funnel through a debris filter to a specimen tube, the debris filter coupled to the funnel and extending into the specimen tube. Further steps include manipulating a reagent cartridge coupled to the funnel to be punctured by one or more protrusions comprising the funnel such that reagent will pass through the debris filter and into the specimen tube.

A further embodiment under the present disclosure is a method for collecting and testing a sample. Steps of the method include receiving a sample in a funnel and directing the sample from the funnel through a debris filter to a specimen tube, the debris filter coupled to the funnel and extending into the specimen tube, wherein the specimen tube is contained by a casing, the casing comprising a top edge comprising an opening configured to receive the specimen tube and a coupler configured to hold a tube cap. Further steps include manipulating a reagent cartridge coupled to the funnel to be punctured by one or more protrusions comprising the funnel such that reagent will pass through the debris filter and into the specimen tube; wherein the funnel and the reagent cartridge comprise a funnel apparatus configured to cover the opening and the coupler of the top edge.

Another embodiment comprises a method of manufacturing a sample collection kit for a specimen tube. Steps include providing a debris filter configured to extend into the specimen tube and to filter test material passing therethrough; providing a funnel coupled to the debris filter and configured to direct test material through the debris filter; providing a reagent cartridge configured to contain a reagent and to be coupled to the funnel; and providing one or more protrusions on the funnel configured to puncture the reagent cartridge when the reagent cartridge is manipulated by a user such that the reagent will pass into the specimen tube.

Another method under the present disclosure is a method of manufacturing a sample collection kit. Steps include providing a casing configured to contain a specimen tube, a top edge of the casing comprising an opening configured to receive the specimen tube therein and a coupler configured to hold a tube cap.

This summary is provided to introduce a selection of concepts in a simplified form that are further described below in the detailed description. This summary is not intended to identify key features or essential features of the claimed subject matter, nor is it intended to be used as an indication of the scope of the claimed subject matter.

The foregoing has outlined rather broadly the features and technical advantages of the present disclosure in order that the detailed description of the embodiments that follows may be better understood. Additional features and advantages of the disclosure will be set forth in the description which follows, and in part will be obvious from the description, or may be learned by the practice of the disclosure. The features and advantages of the disclosure may be realized and obtained by means of the instruments and combinations particularly pointed out in the appended claims. These and other features of the present disclosure will become more fully apparent from the following description and appended claims or may be learned by the practice of the disclosure as set forth hereinafter. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

In order to describe the manner in which the above recited and other advantages and features of the disclosure can be obtained, a more particular description of the disclosure briefly described above will be rendered by reference to specific embodiments thereof, which are illustrated in the appended drawings. It is appreciated that these drawings depict only typical embodiments of the disclosure and are not therefore to be considered to be limiting of its scope. The disclosure will be described and explained with additional specificity and detail through the use of the accompanying drawings in which:

FIGS. 1A-1B show diagrams of sample collection kit embodiments under the present disclosure;

FIGS. 2A-2B show diagrams of sample collection kit embodiments under the present disclosure;

FIGS. 3A-3F show diagrams of sample collection kit embodiments under the present disclosure;

FIG. 4 shows a diagram of a sample collection kit embodiment under the present disclosure;

FIGS. 5A-5F show diagrams of sample collection kit embodiments under the present disclosure;

FIGS. 6A-6D show diagrams of sample collection kit embodiments under the present disclosure;

FIGS. 7A-7B show diagrams of sample collection kit embodiments under the present disclosure;

FIGS. 8A-8B show diagrams of sample collection kit embodiments under the present disclosure;

FIG. 9 shows a diagram of a method embodiment under the present disclosure;

FIG. 10 shows a diagram of a method embodiment under the present disclosure;

FIG. 11 shows a diagram of a method embodiment under the present disclosure; and

FIG. 12 shows a diagram of a method embodiment under the present disclosure.

### DETAILED DESCRIPTION

Before describing various embodiments of the present disclosure in detail, it is to be understood that this disclosure is not limited to the parameters of the particularly exemplified systems, methods, apparatus, products, processes, and/or kits, which may, of course, vary. Thus, while certain embodiments of the present disclosure will be described in detail, with reference to specific configurations, parameters, components, elements, etc., the descriptions are illustrative and are not to be construed as limiting the scope of the claimed invention. In addition, the terminology used herein is for the purpose of describing the embodiments and is not necessarily intended to limit the scope of the claimed invention.

Embodiments under the present disclosure include sample collection kits that are easy to use for the public, compatible with standard laboratory equipment, and do not tip over.

Advantages of the embodiments described herein include:
the device can be pre-assembled for the end user;
pathogen inactivation reagent and/or nucleic acid stabilization reagent is stored in an enclosed area, so it will not spill on users or be accidently ingested;
tube and funnel can be fully integrated into a device that allows the user to set down the device and with a much lower chance of it tipping over and spilling;
the design can be such that if it were to tip over, the sample would not spill out
the design can include a spot for instructions to be visible while using the device and attached to the device, so they are not lost;
the device can be large enough for those with some physical disabilities—such as Parkinson's disease or arthritis—to still be able to grasp and hold the device for collecting saliva;
yet, design can be small enough that a child or someone with small hands would still be able to hold it safely;
the tube and cap design can be standard size, for easily integrating into labs and automated handlers;
the sample funnel can also include a filtration extension, which will help filter out large debris, such as food particles, that can cause inhibition in your downstream applications or cause difficulty to work with in the lab.

One benefit of certain embodiments under the present disclosure is that all the parts required can be integrated into a single consumable. All other sample collection devices on the market come in multiple pieces—putting the burden on the user to figure out what part goes where/when. With the present disclosure, it can pre-assembled and ready to use. The user can have the directions on the device making it easy to know what to do to have a successful sample collection. Also, the parts of the device can be numbered to match the step in the directions, making it very easy to understand what to do at each step.

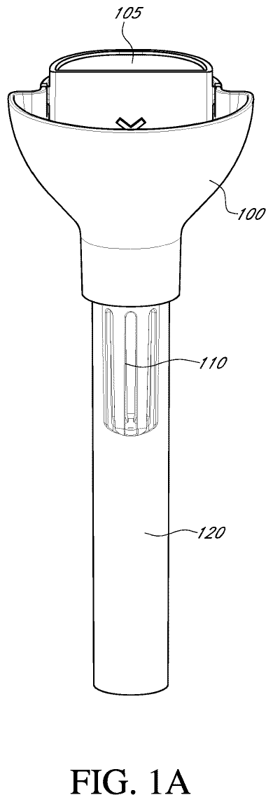

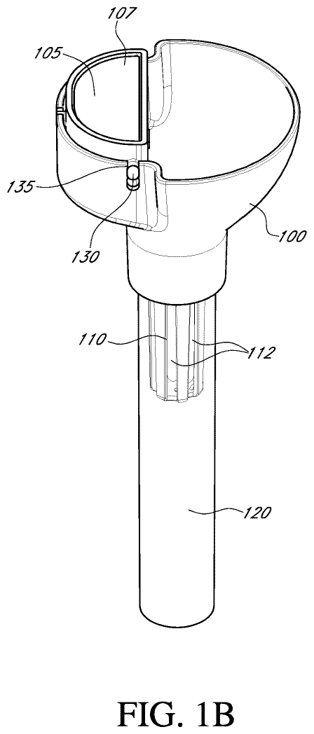

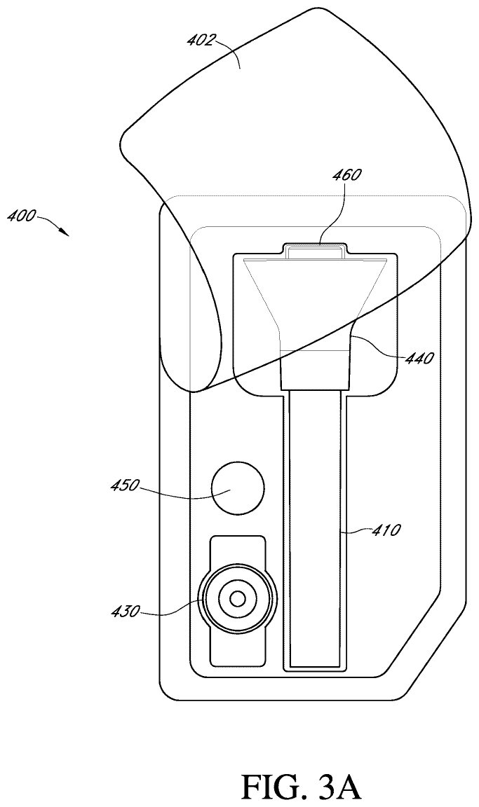

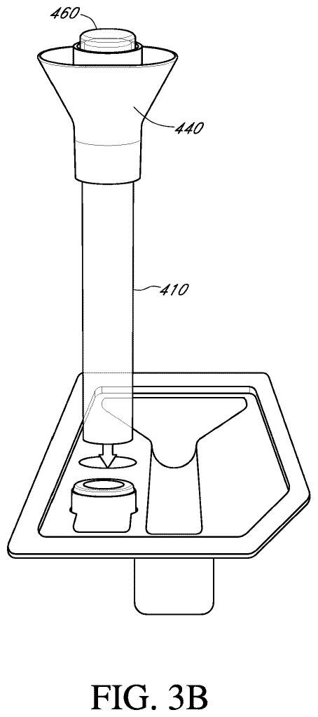

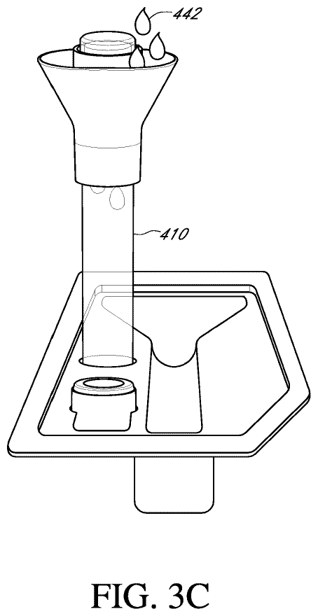

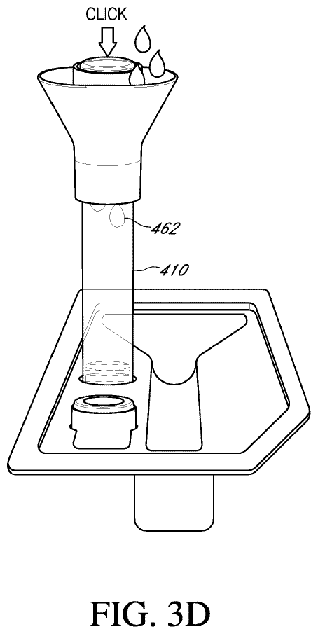

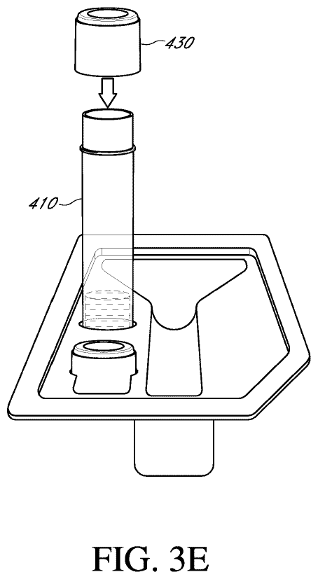

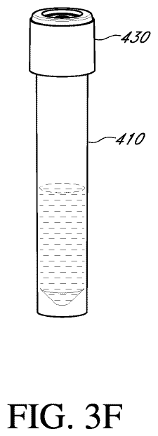

FIGS. 1A-1B show an embodiment of a sample collection kit under the present disclosure. Funnel 100 is detachable from test tube 120. A debris filter 110 (optional) can extend into test tube 120. A reagent cartridge 105 can be coupled to funnel 100, such as via extensions 135 on reagent cartridge 105 and slots 130 on funnel 100. Slits 112 in debris filter 110 can prevent larger particles, such as food, from entering test tube 120. A user can spit into funnel 100 for saliva collection, such as for a COVID-19, influenza, RSV or other virus/bacterial test, human DNA or RNA analysis, or any other types of tests that can be done on saliva samples. Reagent cartridge 105 contains a reagent 107. Protrusions (as shown in FIGS. 3A and 3B) in the funnel bowl and below the reagent cartridge can provide a means of puncturing the reagent cartridge 105. After saliva collection, a user can manipulate the reagent cartridge 105, such as by pressing it down onto the protrusions, breaking the reagent cartridge 105 and causing reagent 107 to flow into the test tube 120. The reagent can assist in the preservation and/or testing of the collected saliva.

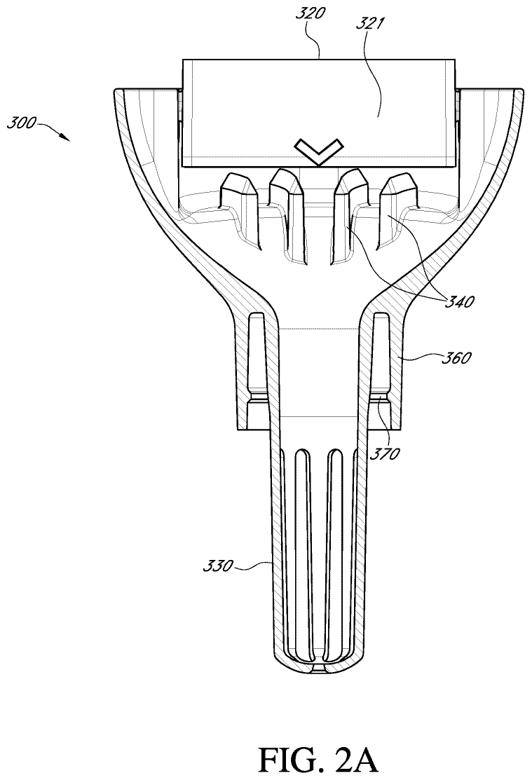

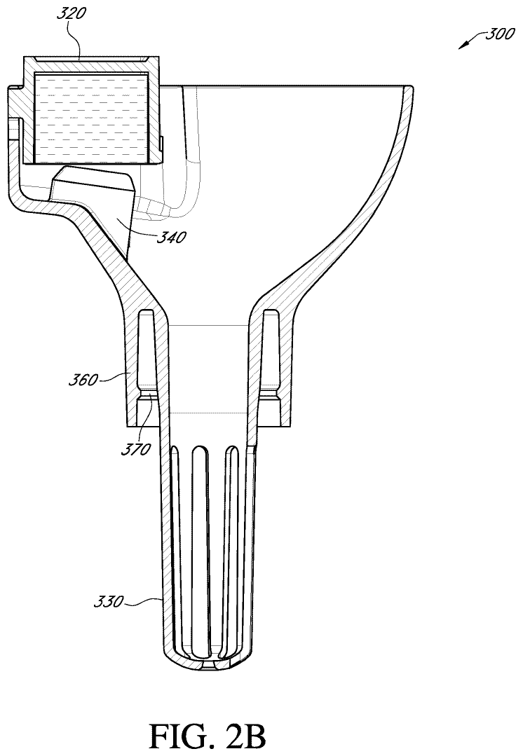

FIGS. 2A-2B show a cut away view of funnel 100 of FIGS. 1A-1B. Funnel 300 has a debris filter 330 extending downward. Inner rim 370 and outer rim 360 extend downward (outer rim 360 further than inner rim 370). Inner and outer rims 370, 360 allow funnel 300 to be detachable from a specimen tube. The rim of a specimen tube can fit between inner and outer rims 370, 360. Reagent cartridge 320 contains a reagent fluid 321. Funnel 300 comprises protrusions 340 beneath reagent cartridge 320. Tabs 327 can assist in holding reagent cartridge 320 in place. Slots 325 on funnel 300 can be coupled to extensions 322 on reagent cartridge 320 to further hold the reagent cartridge 320 in place, while still allowing for vertical movement. A user can manipulate reagent cartridge 320 down onto the protrusions 340 to break it open and release the reagent 321 to flow into a test tube.

FIGS. 3A-3F show further embodiments of a sample collection kit 400. Sample collection kit 400 includes a funnel 440 with a reagent cartridge 460, test tube 410, and tube cap 430. Kit 400 can comprise a plastic (or other material) case with indentations to hold the various components. A tear-off or removable cover 402 can protect kit 400 during shipping and before sale to, or use by, a user. A user can remove funnel 440 and test tube 410 from their indentations in kit 400 and stand the test tube 410 upright in indentation 450 (FIG. 3B). The user can then supply sample 442 through funnel 440 (FIG. 3C). In separate embodiments, reagent cartridge 460 maybe on a side or in the middle of funnel 440. Placing the reagent cartridge toward the middle of the funnel can more evenly distribute mass to allow the test tube 410 to stand upright on a surface without leaning to one side. The user can press down on reagent cartridge 460 to break it open and supply reagent to the test tube 410 (FIG. 3D). The user can then place tube cap 430 on test tube 410 to seal the sample and reagent mixture (FIG. 3E). The test tube 410 can then be supplied to a tester, clinic, sent by mail, or otherwise prepared for testing or analysis (FIG. 3F). The embodiment of funnel 440 in FIGS. 3A-3F does not include a debris filter, though one skilled in the art would understand that a debris filter could be included in funnel 440 without departing from the scope of the concepts described herein.

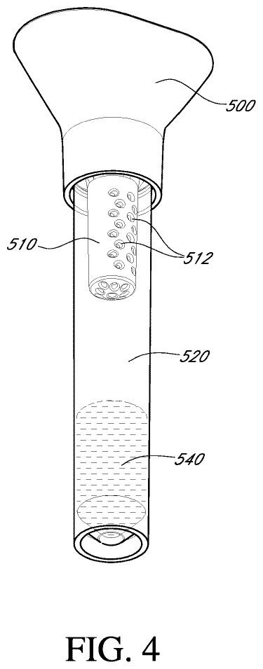

FIG. 4 shows another embodiment of a funnel 500. Funnel 500 is shown coupled to a test tube 320 with reagent 540. This embodiment has a debris filter 510. While some other debris filter embodiments have comprised slits, this embodiment comprises a plurality of apertures or holes 512.

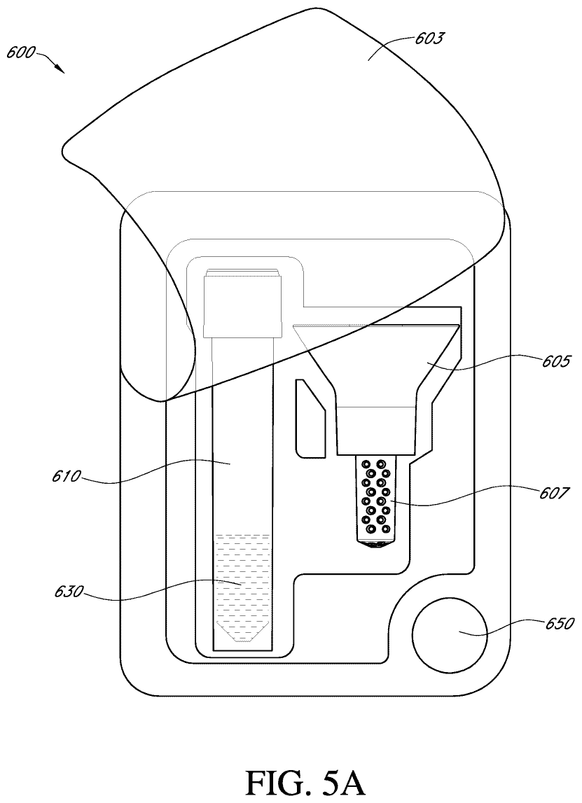

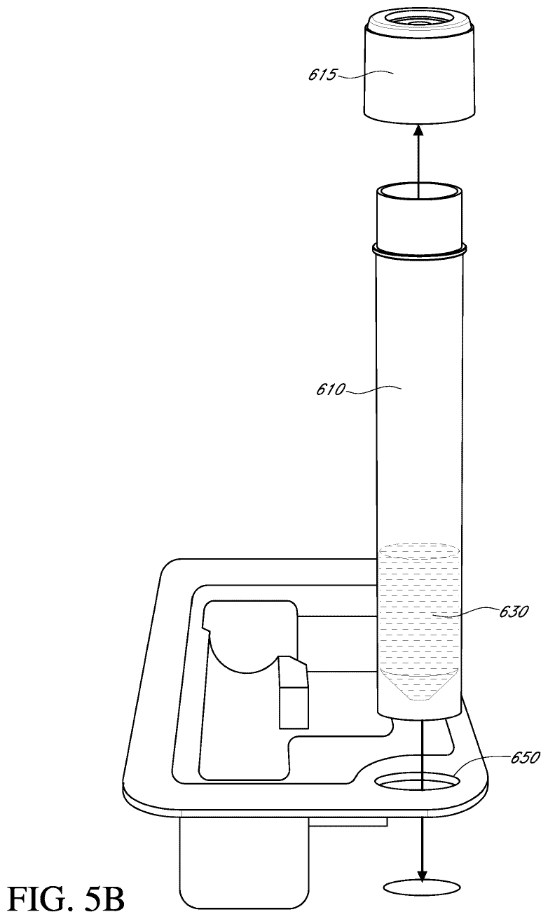

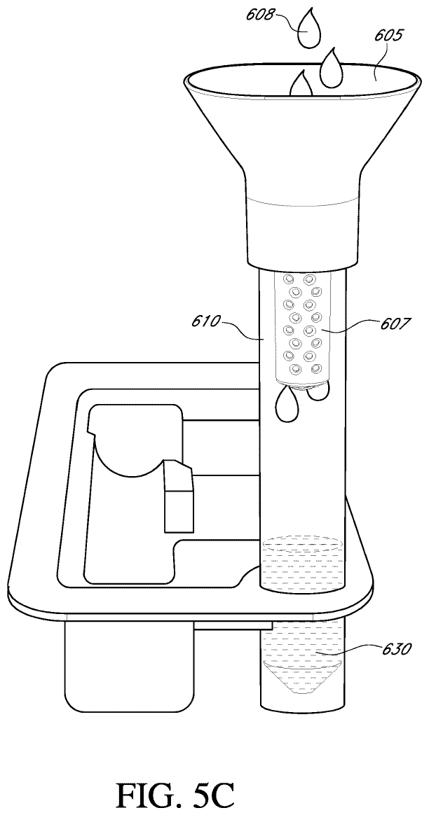

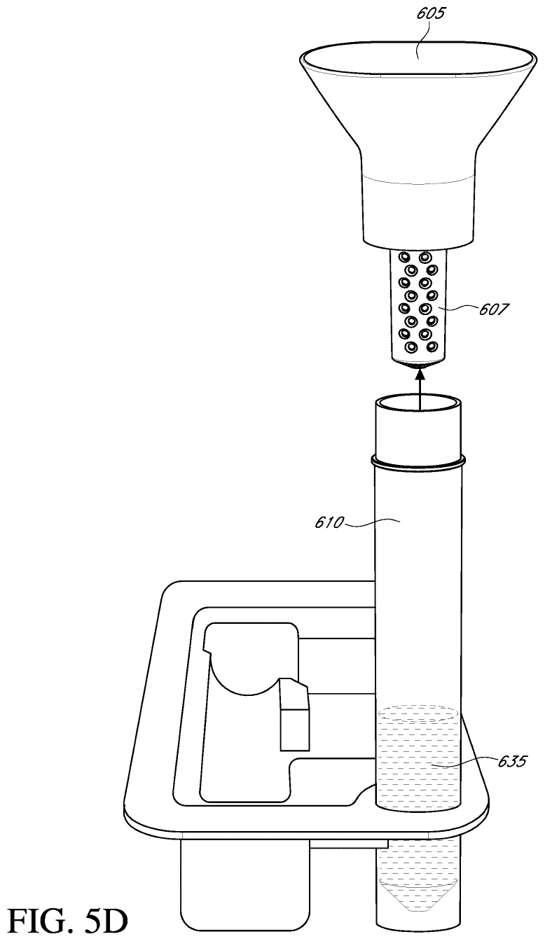

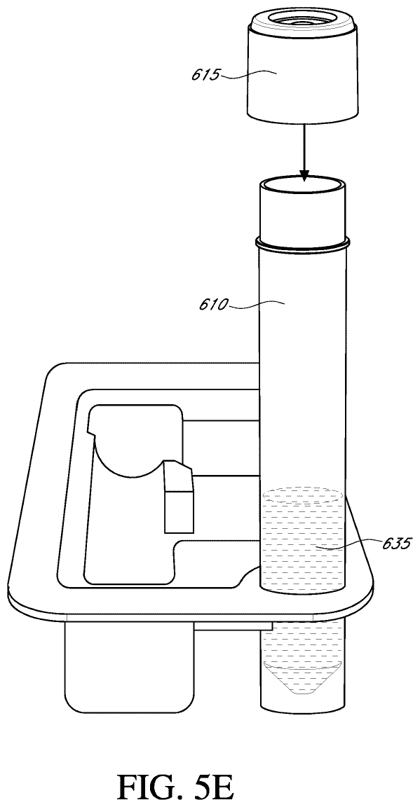

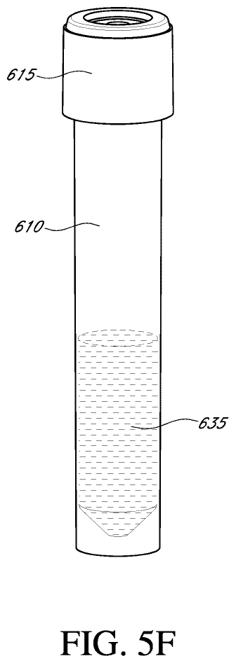

FIGS. 5A-5F show a further embodiment of a sample collection kit 600. Kit 600 is similar to kit 400 of FIGS. 3A-3F, but with several differences. Kit 600 can comprise a plastic (or other material) packaging with a tear off (or removable) cover 603. In kit 600, the test tube 610 can come with reagent 630 already stored therein, meaning there's no reagent cartridge. To contain the reagent 630 the test tube 610 can come with tube cap 615 attached (FIG. 5A). The funnel 605 with debris filter 607 can come detached from test tube 610. Indentations in kit 600 can hold the various components in place. Specific indentation 650 can hold the test tube 610 upright (FIG. 5B). A user can supply sample 608 to filter 605 (FIG. 5C). Debris filter 607 can filter larger material. Sample 608 and reagent 630 combine to form mixture 635 for testing, storage, transport, or analysis (FIG. 5D). Filter 605 can be removed from test tube 610 and tube cap 615 placed back onto test tube 610 (FIG. 5E). The test tube 610 with tube cap 615 and mixture 635 can be removed from kit 600 for analysis or other use (FIG. 5F).

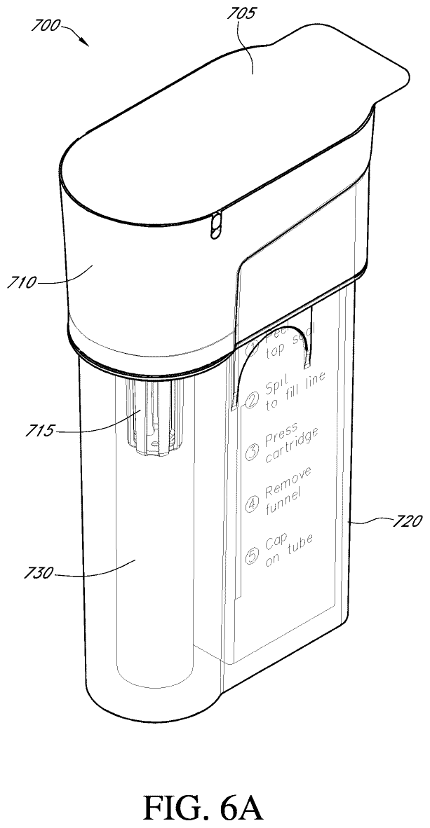

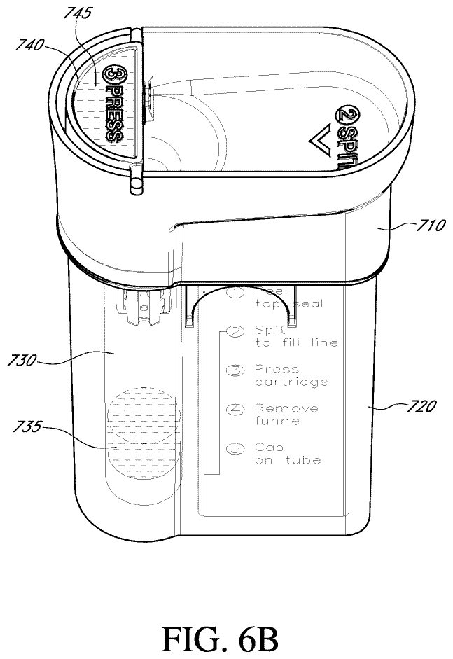

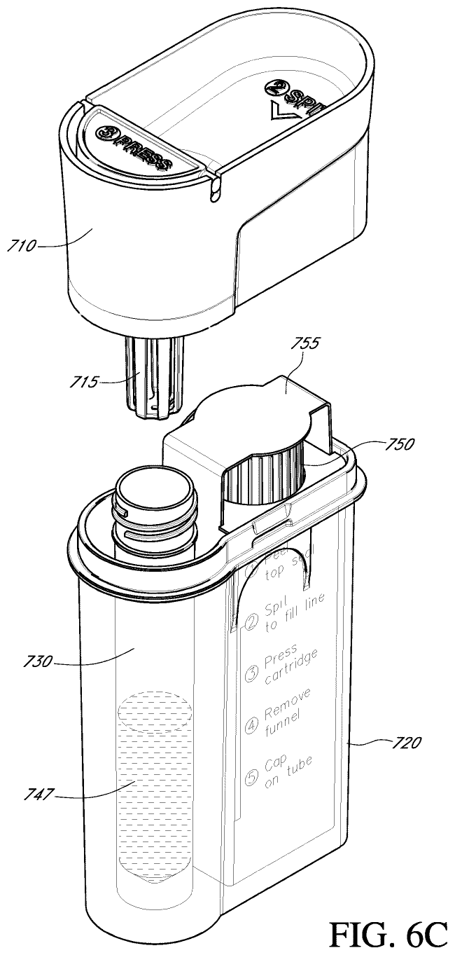

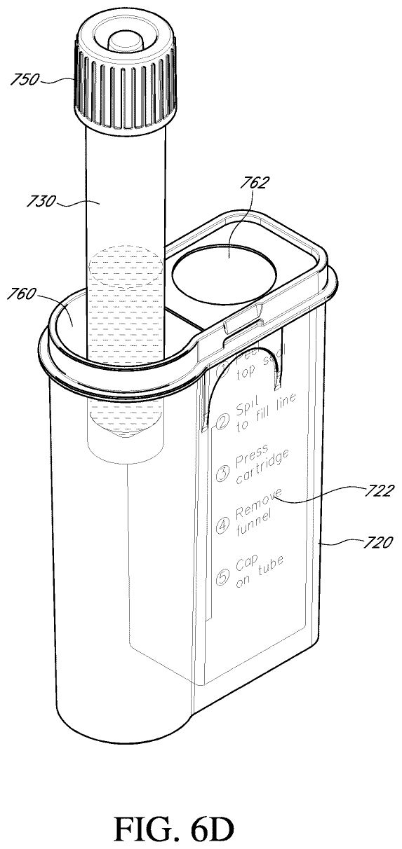

FIGS. 6A-6D show another possible embodiment of a sample collection kit 700. Kit 700 can have tear-off, peel-off, or any other removable cover 705. Funnel 710 can couple to a top edge of a casing 720 with a test tube 730 on one side (FIG. 6A). Removing cover 705 can show a reagent cartridge 740 with reagent 745 and coupled within funnel 710. A user can provide sample 735 to funnel 710. The sample 735 will then pass through debris filter 715 into test tube 730 (FIG. 6B). Reagent cartridge 740 can be manipulated by a user to puncture it and release the reagent 745 into the test tube. Manipulation can be for example, by pressing on top or any part of the reagent container or by any other means such as pushing, twisting or other. Reagent 745 and sample 735 combine into mixture 747 (FIG. 6C). Funnel 710 can be removed from casing 720 and test tube 730. Casing can comprise an opening 760 on a top edge that can receive the test tube 730. Casing can also comprise an indentation or opening 762 that can receive the tube cap 750. A piece of material 755 can help hold tube cap 750 in place on casing and in indentation 762. Tube cap 750 can be coupled to casing 720 via other means: it could press onto a ridge or protrusion, it could be held in place by a plurality of edges or raised protrusions, or other coupling means. After supplying sample 735 and emptying reagent cartridge 740, the test tube 730 can be covered with tube cap 750 and can be removed from casing 720 (FIG. 6D). Testing, analysis, transportation, or other actions can then be performed. Casing 720 can, in some embodiments, comprise writing 722 on a side. This writing can include instruction for sample collection, a fill line for sample, multiple languages, advertisements, or other communications. Casing 720 preferably has a flat bottom edge so that it can stand upright without falling over.

Advantages of the embodiment shown in FIGS. 6A-6D over a simple tube or tube funnel combination can be seen. Kit 700 contains all of the parts and pieces in a single device when assembled without having separate tubes, caps, instructions, or the like. Kit 700 provides an elongated funnel for easier sample collection and casing 720 provides a stable platform to stand kit 700 on a surface for collection or manipulation of reagent cartridge 740, and while removing the funnel and placing the cap 750 on the test tube 730. Kit 700 can also incorporate directions or instruction directly into the device, saving the user from having to locate or keep track of those separately. Tube 730 is easily removed for shipping or processing.

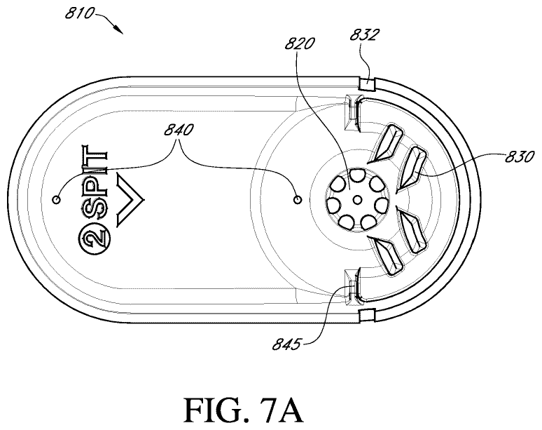

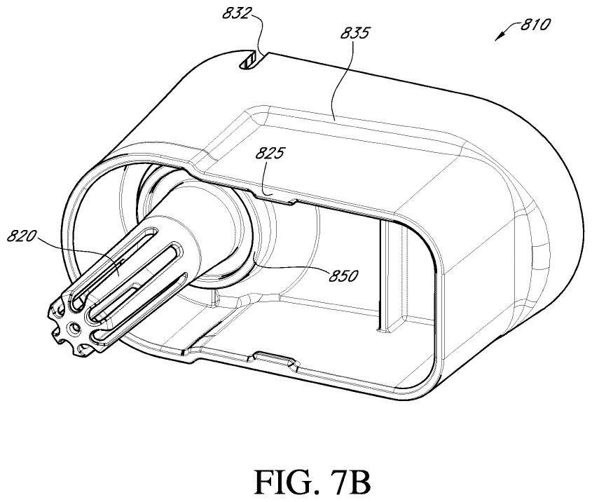

Two different perspective views of funnel 710 of FIGS. 6A-6D are shown in FIGS. 7A-7B. FIG. 7A shows a top-down view of funnel 810 and FIG. 7B shows a bottom perspective view. Funnel 810 has a debris filter 820 extending out the bottom. Inner rim 850 surrounds the debris filter and may be helpful in coupling to a test tube or a casing as shown in FIGS. 6A-6D. Outer rim 825 may engage the outer edge of a casing. Ridge 835 can provide a place for a user to hold onto funnel 810 to remove it from a casing. Slot 832 can couple to extensions on a reagent cartridge. Protrusions 830 help in puncturing a reagent cartridge. Rims 845 can hold a reagent cartridge in place. Vent holes 840 can help in the flow of sample, reagent, or other fluids into the specimen tube below.

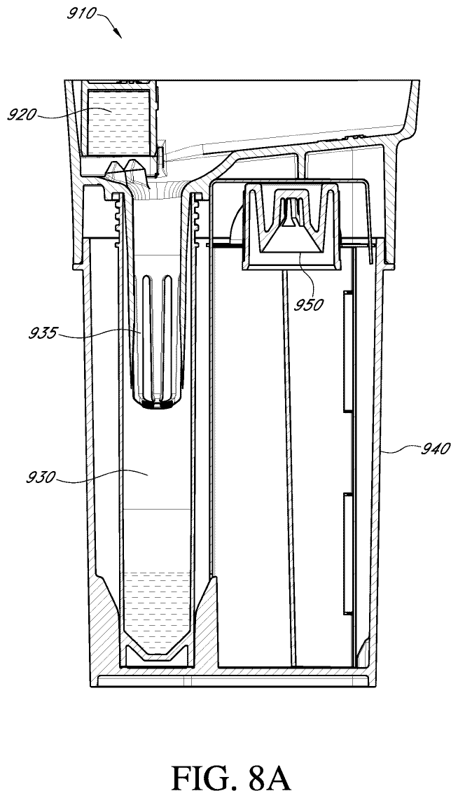

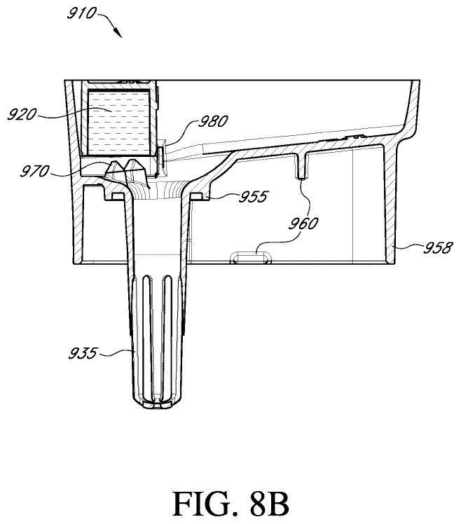

FIGS. 8A-8B show cut away views of a funnel 910. In FIG. 8A, funnel 910 is coupled to casing 940 which contains test tube 930. Debris filter 935 extends into test tube 930 and tube cap 950 is coupled to casing 940. Reagent cartridge 920 is coupled to funnel 910. FIG. 8B shows funnel 910 detached from casing 940. Reagent cartridge 920 is held in place by ridges 980 above protrusions 970 which can puncture reagent cartridge 920. Inner rim 955 surrounds debris filter 935 and can hold a test tube in place. Ridges 960 can be used to couple to casing 940 and to hold funnel 910 in place on casing 940 and above tube cap 950. Outer rim 958 can couple to the outer surface of casing 940.

Debris filter 935 is designed to filter out larger debris or viscous fluids from the sample. Embodiments of the debris filter can take many forms as illustrated in various embodiment described herein. Debris filter 935 as show includes 2 mm slits that extend down the entire portion of the filter below the housing. However, many different configurations are possible. The slits may be sizes other than 2 mm such as 0.5 mm, 1 mm, 1.5 mm, 2.5 mm, or any other appropriate size desired by a user. In addition, the slits may only extend through half of the projection or may extend for more or less. Instead of slits, holes may be incorporated into the funnel projection to perform the same function, as shown in FIGS. 4 and 5A-5F. The holes may be of a single size or a variety of sizes, from for example 0.5 mm to 3 mm or larger, or any appropriate size desired by a user.

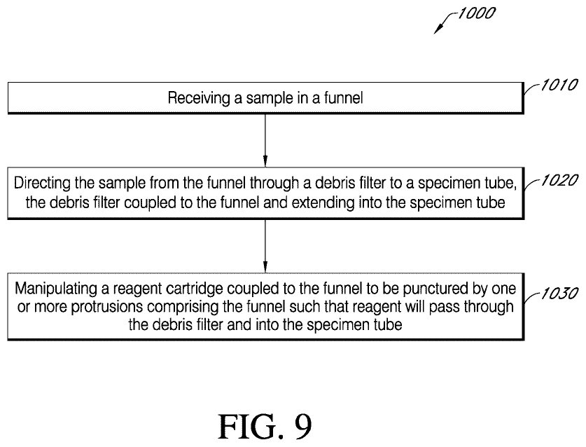

FIG. 9 shows a possible method embodiment 1000 under the present disclosure of collecting and testing a sample. Step 1010 is receiving a sample in a funnel. Step 1020 is directing the sample from the funnel through a debris filter to a specimen tube, the debris filter coupled to the funnel and extending into the specimen tube. Step 1030 is manipulating a reagent cartridge coupled to the funnel to be punctured by one or more protrusions comprising the funnel such that reagent will pass through the debris filter and into the specimen tube.

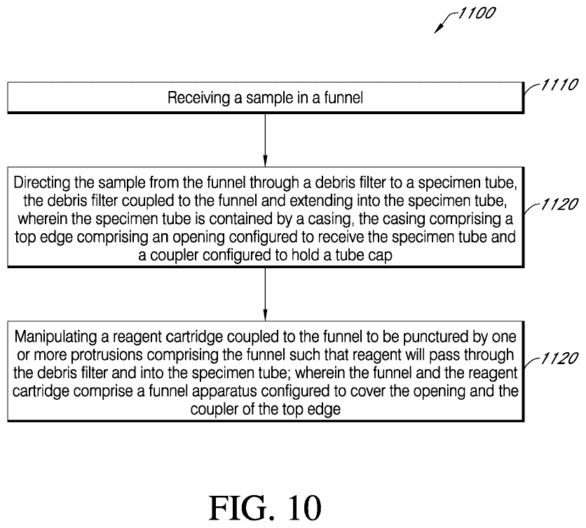

FIG. 10 shows another possible method embodiment 1100 under the present disclosure of collecting a sample. Step 1110 is receiving a sample in a funnel. Step 1120 is directing the sample from the funnel through a debris filter to a specimen tube, the debris filter coupled to the funnel and extending into the specimen tube, wherein the specimen tube is contained by a casing, the casing comprising a top edge comprising an opening configured to receive the specimen tube and a coupler configured to hold a tube cap. Step 1130 is manipulating a reagent cartridge coupled to the funnel to be punctured by one or more protrusions comprising the funnel such that reagent will pass through the debris filter and into the specimen tube; wherein the funnel and the reagent cartridge comprise a funnel apparatus configured to cover the opening and the coupler of the top edge. Optional steps in method 1100 can comprise; removing the funnel apparatus from the casing and the specimen tube; removing the tube cap from the coupler; and placing the tube cap on the test tube.

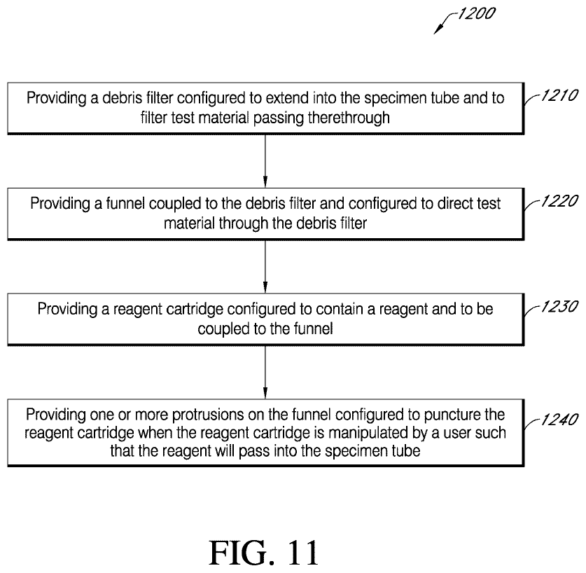

FIG. 11 shows a possible method embodiment 1200 for manufacturing a sample collection kit for a specimen tube. Step 1210 is providing a debris filter configured to extend into the specimen tube and to filter test material passing therethrough. Step 1220 is providing a funnel coupled to the debris filter and configured to direct test material through the debris filter. Step 1230 is providing a reagent cartridge configured to contain a reagent and to be coupled to the funnel. Step 1240 is providing one or more protrusions on the funnel configured to puncture the reagent cartridge when the reagent cartridge is manipulated by a user such that the reagent will pass into the specimen tube.

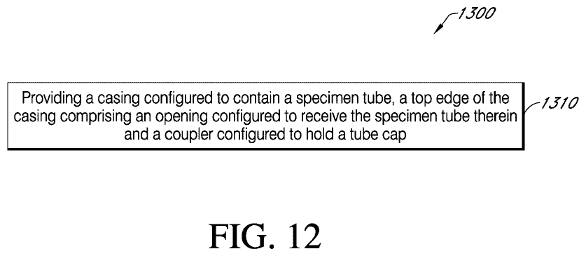

FIG. 12 shows another method embodiment 1300 for manufacturing a sample collection kit. Step 1310 is providing a casing configured to contain a specimen tube, a top edge of the casing comprising an opening configured to receive the specimen tube therein and a coupler configured to hold a tube cap. Further optional steps can include providing a funnel apparatus configured to attach to the top edge of the casing and cover the opening and the coupler. The funnel apparatus can comprise a debris filter configured to extend into the specimen tube and to filter test material passing into the specimen tube; a funnel coupled to the debris filter and configured to receive test material and to direct test material through the debris filter; a reagent cartridge configured to contain a reagent and to be coupled to the funnel; and one or more protrusions on the funnel configured to puncture the reagent cartridge when the reagent cartridge is manipulated by a user such that the reagent will pass into the specimen tube.

The embodiments described herein can be used with any type of tube or receptacle. Test tubes, specimen tubes, storage tubes, of any shape and size, with or without caps, can be used as part of the embodiments described. Sample collection can be of saliva, sputum, mucus, blood, or other bodily fluids or substances.

Embodiments may be with or without reagent. Reagent may be added upon manufacturing, before shipping to users. Alternatively, sample collection kits and embodiments may be manufactured empty and reagent may be added later by labs or other users. Substances added to test or specimen tubes can include reagent or other substances. Such substances can be used for preservation of pathogens, inactivation of pathogens, for preservation of nucleic acids, or for preservation or inactivation of various functionalities or traits. Downstream analysis can be for DNA or RNA (human, viral, bacterial, fungal), protein analysis, analysis of miscellaneous small molecules, general medical examinations, doping tests, and other uses.

### Abbreviated List of Defined Terms

To assist in understanding the scope and content of this written description and the appended claims, a select few terms are defined directly below. Unless defined otherwise, all technical and scientific terms used herein have the same meaning as commonly understood by one of ordinary skill in the art to which the present disclosure pertains.

The terms “approximately,” “about,” and “substantially,” as used herein, represent an amount or condition close to the specific stated amount or condition that still performs a desired function or achieves a desired result. For example, the terms “approximately,” “about,” and “substantially” may refer to an amount or condition that deviates by less than 10%, or by less than 5%, or by less than 1%, or by less than 0.1%, or by less than 0.01% from a specifically stated amount or condition.

Various aspects of the present disclosure, including devices, systems, and methods may be illustrated with reference to one or more embodiments or implementations, which are exemplary in nature. As used herein, the term “exemplary” means “serving as an example, instance, or illustration,” and should not necessarily be construed as preferred or advantageous over other embodiments disclosed herein. In addition, reference to an “implementation” of the present disclosure or invention includes a specific reference to one or more embodiments thereof, and vice versa, and is intended to provide illustrative examples without limiting the scope of the invention, which is indicated by the appended claims rather than by the following description.

As used in the specification, a word appearing in the singular encompasses its plural counterpart, and a word appearing in the plural encompasses its singular counterpart, unless implicitly or explicitly understood or stated otherwise. Thus, it will be noted that, as used in this specification and the appended claims, the singular forms “a,” “an” and “the” include plural referents unless the context clearly dictates otherwise. For example, reference to a singular referent (e.g., “a widget”) includes one, two, or more referents unless implicitly or explicitly understood or stated otherwise. Similarly, reference to a plurality of referents should be interpreted as comprising a single referent and/or a plurality of referents unless the content and/or context clearly dictate otherwise. For example, reference to referents in the plural form (e.g., “widgets”) does not necessarily require a plurality of such referents. Instead, it will be appreciated that independent of the inferred number of referents, one or more referents are contemplated herein unless stated otherwise.

As used herein, directional terms, such as “top,” “bottom,” “left,” “right,” “up,” “down,” “upper,” “lower,” “proximal,” “distal,” “adjacent,” and the like are used herein solely to indicate relative directions and are not otherwise intended to limit the scope of the disclosure and/or claimed invention.

### Conclusion

It is understood that for any given component or embodiment described herein, any of the possible candidates or alternatives listed for that component may generally be used individually or in combination with one another, unless implicitly or explicitly understood or stated otherwise. Additionally, it will be understood that any list of such candidates or alternatives is merely illustrative, not limiting, unless implicitly or explicitly understood or stated otherwise.

In addition, unless otherwise indicated, numbers expressing quantities, constituents, distances, or other measurements used in the specification and claims are to be understood as being modified by the term “about,” as that term is defined herein. Accordingly, unless indicated to the contrary, the numerical parameters set forth in the specification and attached claims are approximations that may vary depending upon the desired properties sought to be obtained by the subject matter presented herein. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the subject matter presented herein are approximations, the numerical values set forth in the specific examples are reported as precisely as possible. Any numerical values, however, inherently contain certain errors necessarily resulting from the standard deviation found in their respective testing measurements.

Any headings and subheadings used herein are for organizational purposes only and are not meant to be used to limit the scope of the description or the claims.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention itemed. Thus, it should be understood that although the present invention has been specifically disclosed in part by preferred embodiments, exemplary embodiments, and optional features, modification and variation of the concepts herein disclosed may be resorted to by those skilled in the art, and such modifications and variations are considered to be within the scope of this invention as defined by the appended items. The specific embodiments provided herein are examples of useful embodiments of the present invention and various alterations and/or modifications of the inventive features illustrated herein, and additional applications of the principles illustrated herein that would occur to one skilled in the relevant art and having possession of this disclosure, can be made to the illustrated embodiments without departing from the spirit and scope of the invention as defined by the items and are to be considered within the scope of this disclosure.

It will also be appreciated that systems, devices, products, kits, methods, and/or processes, according to certain embodiments of the present disclosure may include, incorporate, or otherwise comprise properties or features (e.g., components, members, elements, parts, and/or portions) described in other embodiments disclosed and/or described herein. Accordingly, the various features of certain embodiments can be compatible with, combined with, included in, and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include said features, members, elements, parts, and/or portions without necessarily departing from the scope of the present disclosure.

Moreover, unless a feature is described as requiring another feature in combination therewith, any feature herein may be combined with any other feature of a same or different embodiment disclosed herein. Furthermore, various well-known aspects of illustrative systems, methods, apparatus, and the like are not described herein in particular detail in order to avoid obscuring aspects of the example embodiments. Such aspects are, however, also contemplated herein.

All references cited in this application are hereby incorporated in their entireties by reference to the extent that they are not inconsistent with the disclosure in this application. It will be apparent to one of ordinary skill in the art that methods, devices, device elements, materials, procedures, and techniques other than those specifically described herein can be applied to the practice of the invention as broadly disclosed herein without resort to undue experimentation. All art-known functional equivalents of methods, devices, device elements, materials, procedures, and techniques specifically described herein are intended to be encompassed by this invention.

When a group of materials, compositions, components, or compounds is disclosed herein, it is understood that all individual members of those groups and all subgroups thereof are disclosed separately. When a Markush group or other grouping is used herein, all individual members of the group and all combinations and sub-combinations possible of the group are intended to be individually included in the disclosure. Every formulation or combination of components described or exemplified herein can be used to practice the invention, unless otherwise stated. Whenever a range is given in the specification, for example, a temperature range, a time range, or a composition range, all intermediate ranges and subranges, as well as all individual values included in the ranges given are intended to be included in the disclosure. All changes which come within the meaning and range of equivalency of the items are to be embraced within their scope.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A sample collection kit for a specimen tube, comprising:
a funnel detachably coupled to the storage tube and configured to direct test material into the specimen tube;
a reagent cartridge configured to contain a reagent and to be coupled to the funnel; and
one or more protrusions on the funnel configured to puncture the reagent cartridge when the reagent cartridge is manipulated by a user such that the reagent will pass into the specimen tube.

2. The collection kit of claim 1 further comprising a debris filter configured to extend into the specimen tube from the funnel and to filter test material passing therethrough

3. The collection kit of claim 2 further comprising an inner rim and an outer rim around the debris filter, the outer rim longer than the inner rim and the inner rim and outer rim configured to hold the sample collection kit in place on a storage tube.

4. The collection kit of any of claims 1 to 3, further comprising;
the specimen tube;
a tube cap; and
a package, the package comprising one or more recessed portions configured to hold the sample collection kit, specimen tube and tube cap, and further configured to hold the storage tube upright for collection of test material by a user.

5. The collection kit of any of claims 2 to 4 wherein the debris filter comprises one or more slits.

6. The collection kit of any of claims 2 to 4 wherein the debris filter comprises a plurality of holes.

7. The collection kit of claim 5 wherein the one or more slits are 2 mm wide.

8. The collection kit of claim 5 wherein the one or more slits are 1 mm wide.

9. The collection kit of any of claims 1 to 8 wherein the funnel comprises one or more slots, the reagent cartridge comprises one or more extensions, and the reagent cartridge can be coupled to the funnel with the one or more slots and one or more extensions.

10. The collection kit of claim 9 wherein the one or more slots and one or more extensions hold the reagent cartridge on a side of the funnel.

11. The collection kit of any of claims 1 to 9 wherein the reagent cartridge is held in the middle of the funnel.

12. The collection kit of claim 1 further comprising a casing to hold the specimen tube and funnel, such that the casing provides a stable platform for the specimen tube and funnel to stand upright on a surface.

13. A sample collection kit for a specimen tube, comprising:
a funnel configured to receive a sample from a user;
wherein the funnel is configured to be attachable to a specimen tube.

14. The sample collection kit of claim 13 further comprising a debris filter coupled to the funnel and configured to receive the sample therefrom, the debris filter configured to extend into a specimen tube.

15. The collection kit of claim 13 further comprising the storage specimen tube and a reagent within the reagent cartridge.

16. The collection kit of any of claims 13 to 15, further comprising a rim around the debris filter, the rim configured to hold the sample collection kit in place on a specimen tube

17. The collection kit of any of claims 13 to 15, further comprising an inner rim and an outer rim around the debris filter, the outer rim longer than the inner rim and the inner rim and outer rim configured to hold the sample collection kit in place on a specimen tube.

18. A sample collection kit for a specimen tube, comprising:
a casing configured to contain a specimen tube, a top edge of the casing comprising an opening configured to receive the specimen tube therein and a coupler configured to hold a tube cap.

19. The collection kit of claim 18, further comprising:
a funnel apparatus configured to attach to the top edge of the casing and cover the opening and the coupler, and comprising;
a debris filter configured to extend into the specimen tube and to filter test material passing into the specimen tube;
a funnel coupled to the debris filter and configured to receive test material and to direct test material through the debris filter;
a reagent cartridge configured to contain a reagent and to be coupled to the funnel; and
one or more protrusions on the funnel configured to puncture the reagent cartridge when the reagent cartridge is manipulated by a user such that the reagent will pass into the specimen tube.

20. A sample collection kit for a specimen tube, comprising:
a funnel detachably coupled to the specimen tube and configured to direct test material into the specimen tube;
a reagent cartridge configured to contain a reagent and to be coupled to the funnel; and
one or more protrusions on the funnel configured to puncture the reagent cartridge when the reagent cartridge is manipulated by a user such that the reagent will pass into the specimen tube;
wherein the funnel and one or more protrusions comprise a funnel apparatus configured to attach to a top edge of a casing, and further configured to cover an opening for a specimen tube and a coupler for a tube cap on the top edge.

21. The collection kit of any claims 18 to 20, further comprising a debris filter configured to extend into the specimen tube and to filter test material passing therethrough.

22. The collection kit of any of claims 18 to 19 further comprising a tube cap coupled to the coupler.

23. The collection kit of any of claims 18 to 22 wherein the funnel apparatus comprises a peel-off cover.

24. The collection kit of any of claims 18 to 23 wherein the debris filter comprises one or more slits.

25. The collection kit of any of claims 18 to 23 wherein the debris filter comprises a plurality of holes.

26. The collection kit of claim 24 wherein the one or more slits are 2 mm wide.

27. The collection kit of claim 24 wherein the one or more slits are 1 mm wide.

28. The collection kit of any of claims 18 to 27 wherein the funnel comprises one or more slots, the reagent cartridge comprises one or more extensions, and the reagent cartridge can be coupled to the funnel with the one or more slots and one or more extensions.

29. The collection kit of claim 28 wherein the one or more slots and one or more extensions hold the reagent cartridge on a side of the funnel.

30. The collection kit of any of claims 18 to 28 wherein the reagent cartridge is held in the middle of the funnel.

31. The collection kit of any of claims 18 to 30 wherein the casing comprises printed material on a surface.

32. The collection kit of claim 31 wherein the printed material comprises instructions for use.

33. The collection kit of claim 31 or 32 wherein the printed material comprises a fill level.

34. The collection kit of any of claims 18 to 33 wherein the casing comprises a flat bottom edge.

35. The collection kit of any of claims 18 to 34, further comprising a rim around the debris filter, the rim configured to hold the sample collection kit in place on a specimen tube

36. The collection kit of any of claims 18 to 34, further comprising an inner rim and an outer rim around the debris filter, the outer rim longer than the inner rim and the inner rim and outer rim configured to hold the sample collection kit in place on a specimen tube.

37. A method of collecting and testing a sample, comprising;
receiving a sample in a funnel;
directing the sample from the funnel through a debris filter to a specimen tube, the debris filter coupled to the funnel and extending into the specimen tube; and
manipulating a reagent cartridge coupled to the funnel to be punctured by one or more protrusions comprising the funnel such that reagent will pass through the debris filter and into the specimen tube.

38. A method of collecting a sample, comprising:
receiving a sample in a funnel;
directing the sample from the funnel through a debris filter to a specimen tube, the debris filter coupled to the funnel and extending into the specimen tube, wherein the specimen tube is contained by a casing, the casing comprising a top edge comprising an opening configured to receive the specimen tube and a coupler configured to hold a tube cap; and
manipulating a reagent cartridge coupled to the funnel to be punctured by one or more protrusions comprising the funnel such that reagent will pass through the debris filter and into the specimen tube;
wherein the funnel and the reagent cartridge comprise a funnel apparatus configured to cover the opening and the coupler of the top edge.

39. The method of claim 38 further comprising;
removing the funnel apparatus from the casing and the specimen tube;
removing the tube cap from the coupler; and
placing the tube cap on the specimen tube.

40. A method of manufacturing a sample collection kit for a specimen tube, comprising:
providing a debris filter configured to extend into the storage tube and to filter test material passing therethrough;
providing a funnel coupled to the debris filter and configured to direct test material through the debris filter;
providing a reagent cartridge configured to contain a reagent and to be coupled to the funnel; and
providing one or more protrusions on the funnel configured to puncture the reagent cartridge when the reagent cartridge is manipulated by a user such that the reagent will pass into the specimen tube.

41. The method of claim 40 further comprising putting reagent in the reagent cartridge prior to transport.

42. The method of claim 40 further comprising transporting the sample collection kit prior to putting reagent in the reagent cartridge.

43. A method of manufacturing a sample collection kit, comprising:
providing a casing configured to contain a specimen tube, a top edge of the casing comprising an opening configured to receive the specimen tube therein and a coupler configured to hold a tube cap.

44. The method of claim 43, further comprising:
providing a funnel apparatus configured to attach to the top edge of the casing and cover the opening and the coupler, and comprising;
a debris filter configured to extend into the test tube and to filter test material passing into the specimen tube;
a funnel coupled to the debris filter and configured to receive test material and to direct test material through the debris filter;
a reagent cartridge configured to contain a reagent and to be coupled to the funnel; and
one or more protrusions on the funnel configured to puncture the reagent cartridge when the reagent cartridge is manipulated by a user such that the reagent will pass into the specimen tube.

45. The sample collection kit of any of claims 1 to 36 wherein the sample collection kit is configured for downstream analysis of at least one of the following: DNA, RNA, human, viral, bacterial, fungal, protein, small molecule, general medical examination, and doping.
